(19) United States
(12) Patent Application Publication
(10) Pub. No.: US 2018/0335383 A1
(43) Pub. Date: Nov. 22, 2018

(54) METHOD AND APPARATUS FOR DETECTING THE CONTENT OF OLEIC ACID BY OPTICAL FIBER, AND MANUFACTURING METHOD OF THE APPARATUS

(71) Applicant: MING CHUAN UNIVERSITY, TAIPEI (TW)

(72) Inventors: YU-CHENG LIN, TAIPEI (TW); LIANG-YU CHEN, TAIPEI (TW); BING-RU LI, TAIPEI (TW); CHUN WANG, TAIPEI (TW); SHAO-TING LIN, TAIPEI (TW)

(21) Appl. No.: 15/687,718

(22) Filed: Aug. 28, 2017

(30) Foreign Application Priority Data
May 17, 2017 (TW) 106116233

(51) Int. Cl. G01N 21/552; G01N 33/03

(52) U.S. Cl. CPC G01N 21/554; G01N 2201/088; G01N 33/03

(57) ABSTRACT

Disclosed are a method and apparatus for detecting the content of oleic acid by optical fiber, and manufacturing method of the apparatus. By the principle of Surface Plasmon Resonance (SPR), a metal layer in a sensing region excites an analyte of oleic acid to produce a plasmon resonance, and a physical property of the plasmon resonance such as a resonant wavelength is measured to define the concentration of oleic acid of the analyte, so that the present invention can measure the concentration of oleic acid accurately and quickly to reduce the time and cost of detecting the oleic acid effectively, so as to improve the efficiency of detecting the oleic acid.

### FIELD OF THE INVENTION

The present invention relates to a method and apparatus for detecting the content of oleic acid by optical fiber and a manufacturing method of the apparatus, and more particularly to the method and apparatus of defining the concentration of oleic acid of an analyte by measuring a physical property of the analyte based on the principle of Surface Plasmon Resonance (SPR) of the optical fiber when the plasmon resonance occurs.

### BACKGROUND OF THE INVENTION

Vegetable oil contains a small amount of oleic acid. As vegetable oil is heated, the oleic acid in oil will generate free fatty acids (FFAs) owing to oxidation, thermal reaction and hydrolysis. After human eats and absorbs free fatty acids, the free fatty acids in the blood circulating system will cause atherosclerosis and also an increased risk of high blood pressure.

In recent years, food safety becomes an issue that demands immediate attention and requires feasible solutions, and the events of poor-quality oils occur frequently, and edible oils may be refreshed periodically by boiling the oils at high temperature repeatedly. As a result, the edible oils are accumulated with a large quantity of substances harmful to human health. Related researches indicate that the quality of edible oils is related to the content of oleic acid. Therefore, the quality of edible oils can be evaluated by detecting the concentration of oleic acid of the edible oils.

In a conventional method of detecting oils, an acid value test paper is used for detecting free fatty acids, and a color change of the acid value test paper is compared with a standard color chart to evaluate the quality of oils. Although the response of such test is quick, it is difficult to compare a color with a color chart accurately, so that there may be errors in the detected result. In addition, FIG. 1 shows a color change of the acid value test paper, and the range of detection is limited to a free fatty acid concentration below 2.5%, and any concentration above 2.5% cannot be detected.

Through the test conducted by an acid-alkali neutralization titration, the pH value of an oil can be detected and measured, but a relatively larger quantity of the analyte is required, and the detection process is time-consuming and laborious. Obviously, this method is not suitable for instant quantitative measurements.

In another conventional detection method, gas chromatography-mass spectrometry is used for detecting oils, and this method is capable of providing a qualitative and quantitative analysis, but the equipment is very expensive, and the involved compounds may affect the test result, so that the applicability of this method is limited.

In view of the aforementioned drawbacks of the conventional methods and apparatuses for detecting the content of oleic acid, the inventor of the present invention conducted extensive research and development on the detection of oleic acid and finally provided feasible solutions in accordance with the present invention to overcome the drawbacks of the prior art.

### SUMMARY OF THE INVENTION

Therefore, it is a primary objective of the present invention to overcome the aforementioned drawbacks of the prior art by providing a method for detecting the content of oleic acid by optical fiber, and the method comprises: setting a metal layer in a sensing region and an analyte onto the metal layer; applying a transverse magnetic wave of a light beam into the sensing region through a light guide medium; letting the light beam excite the metal layer and the analyte to produce a plasmon resonance; measuring at least one physical property of the plasmon resonance; and defining the concentration of oleic acid of the analyte according to the physical property.

In the method for detecting the content of oleic acid by optical fiber, the metal layer is a gold (Au), silver (Ag), aluminum (Al) or copper (Cu) thin film.

In the method for detecting the content of oleic acid by optical fiber, the metal layer has a thickness falling within a range from 40 nm to 60 nm.

In the method for detecting the content of oleic acid by optical fiber, the analyte is a solution including oleic acid and alcohol solvent.

In the method for detecting the content of oleic acid by optical fiber, the alcohol solvent is ethanol.

In the method for detecting the content of oleic acid by optical fiber, the light beam has a wavelength falling within a range from 400 nm to 1800 nm before the light beam is incident onto the sensing region.

In the method for detecting the content of oleic acid by optical fiber, the physical property of the plasmon resonance can be measured by an optical spectrum analyzer.

In the method for detecting the content of oleic acid by optical fiber, the light guide medium has an optical fiber body, and the optical fiber body has a center layer and a cladding layer, and the cladding layer is coaxial with the center layer, and the optical fiber body has a groove formed on a side of the middle of the optical fiber body and sunken into the center layer, and the bottom wall of the groove forms the sensing region, and the light beam is incident from an end of the center layer and transmitted to the sensing region.

In the method for detecting the content of oleic acid by optical fiber, the bottom wall of the groove is a planar surface.

In the method for detecting the content of oleic acid by optical fiber, the metal layer is formed at the sensing region by sputtering.

In the method for detecting the content of oleic acid by optical fiber, the physical property of the plasmon resonance is the resonant wavelength when the plasmon resonance occurs.

The present invention further provides an apparatus for detecting the content of oleic acid by optical fiber, and the apparatus applies the aforementioned method for detecting the content of oleic acid by optical fiber and comprises a light guide medium having an optical fiber body, wherein the optical fiber body has a center layer and a cladding layer, and the cladding layer is coaxial with the center layer, and the optical fiber body has a groove formed on a side of the middle of the optical fiber body and sunken into the center layer, and the bottom wall of the groove forms the sensing region, and the sensing region has the metal layer.

In the apparatus for detecting the content of oleic acid by optical fiber, the bottom wall of the groove is a planar surface.

In the apparatus for detecting the content of oleic acid by optical fiber, the metal layer is formed at the sensing region by sputtering.

In the apparatus for detecting the content of oleic acid by optical fiber, the groove at the center layer has a depth approximately equal to half of the diameter of the center layer.

In the apparatus for detecting the content of oleic acid by optical fiber, the cladding layer has an external diameter of 125 μm, and the center layer has an external diameter of 62.5 μm, and the groove has an axial length of 5 mm, and the groove has a depth of 62.5 μm.

In the apparatus for detecting the content of oleic acid by optical fiber, the optical fiber body is a glass optical fiber.

The present invention further provides a manufacturing method of an apparatus for detecting the content of oleic acid by optical fiber, and the manufactured method is used for manufacturing the aforementioned apparatus for detecting the content of oleic acid by optical fiber. The manufacturing method comprises the steps of: removing the portion of the cladding layer from a side of the middle of the optical fiber body; performing a rough polishing to the center layer wherein the cladding layer is removed to form the groove; performing a fine polishing of the bottom of the groove to form the planar sensing region; and depositing and forming the metal layer at the sensing region by sputtering.

In summation of the description above, the present invention has the following advantages and effects: The present invention adopts the principle of the Surface Plasmon Resonance (SPR) and uses the physical property of the plasmon resonance to measure the concentration of oleic acid of the analyte to achieve the effects of instant detection, high accuracy, and high sensitivity, and the whole detection process is more stable and not easy to be affected by external factors, and such method and apparatus are advantageous for quantifying the instant detection. In addition, the structure of the optical fiber body and the manufacturing method of the present invention incur a lower cost and a simpler and easier manufacture than those of the conventional SPR fiber-optic sensor. The apparatus of the invention requires no angle calibration and it can be connected to an optical spectrum analyzer directly and also connected to the optical fiber, and the spectrum can be used for observing a change of the SPR signals to obtain the concentration of oleic acid immediately.

### BRIEF DESCRIPTION OF THE DRAWINGS

FIG. 1 is a schematic view showing a conventional experiment of using an acid value test paper to measure a free fatty acid concentration ranging from 1% to 6%;

FIG. 2 is a flow chart of a method for detecting the content of oleic acid by optical fiber in accordance with the present invention;

FIG. 3 is a perspective view of an apparatus for detecting the content of oleic acid by optical fiber in accordance with the present invention;

FIG. 4 is a flow chart of a manufacturing method of an apparatus for detecting the content of oleic acid by optical fiber in accordance with the present invention;

FIG. 5 shows the experiment results of oleic acid dissolved in an alcohol solvent in accordance with the present invention by a graph of concentration versus index of refraction;

FIG. 6 shows the experiment result of the present invention by a graph of normalized light intensity of different concentration of oleic acids versus resonant wavelength in a SPR spectrum; and

FIG. 7 is a partial blowup view of a normalized light intensity approaching to zero as depicted in FIG. 6.

### DESCRIPTION OF THE PREFERRED EMBODIMENTS

The technical contents of the present invention will become apparent with the detailed description of preferred embodiments accompanied with the illustration of related drawings as follows. It is intended that the embodiments and figures disclosed herein are to be considered illustrative rather than restrictive.

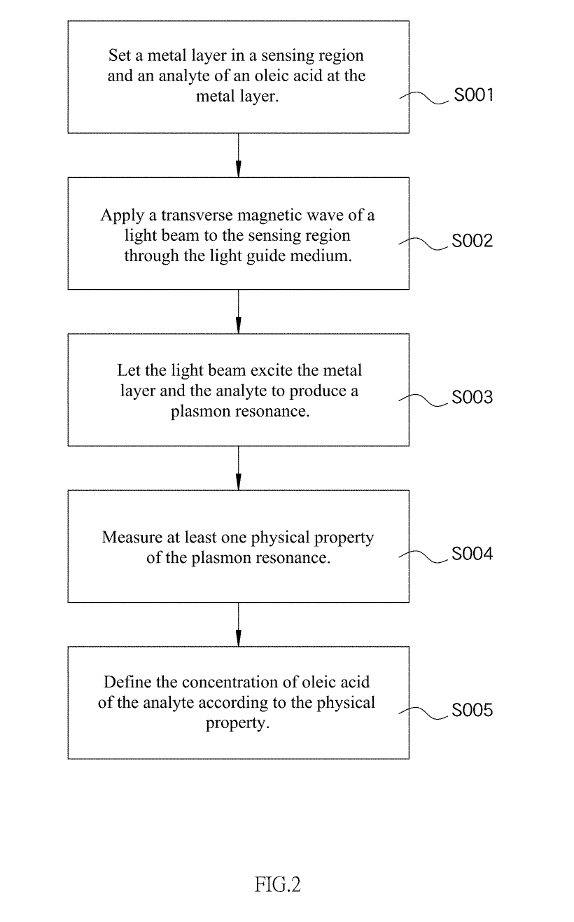

With reference to FIG. 2 for a method for detecting the content of oleic acid by optical fiber in accordance the present invention, the method comprises the following steps:

S001: Set a metal layer 2 in a sensing region 1 and an analyte of an oleic acid at the metal layer 2.

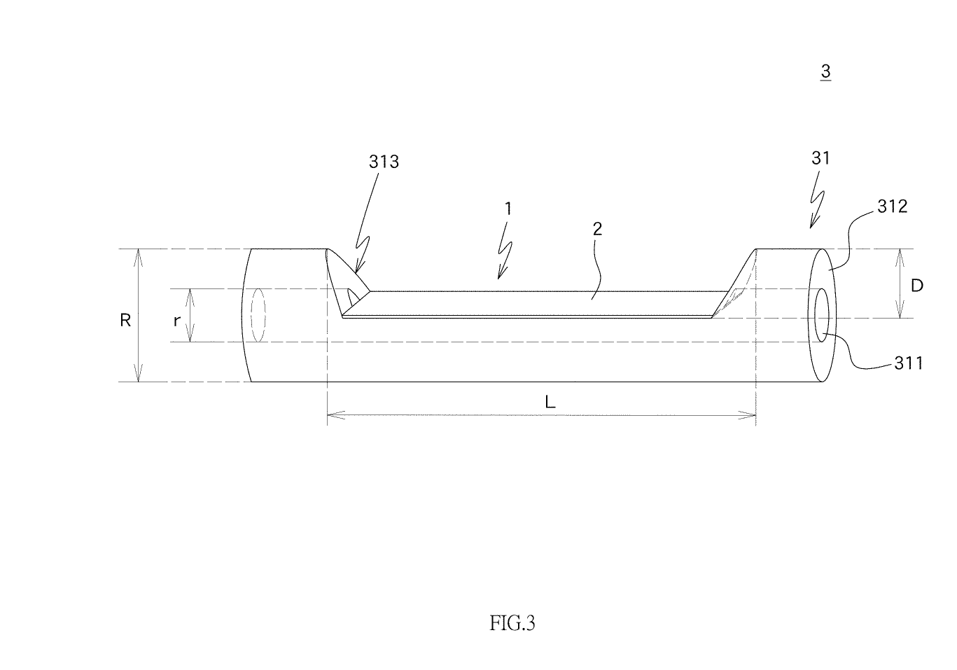

In a specific preferred embodiment as shown in FIG. 3, the sensing region 1 is disposed at an apparatus for detecting the content of oleic acid by optical fiber and comprises: a light guide medium 3 having an optical fiber body 31, wherein the optical fiber body 31 has a center layer 311 and a cladding layer 312, and the cladding layer 312 is coaxial with the center layer 311, and a groove 313 is formed on a side of the middle of the optical fiber body 31 and sunken into the center layer 311, and the bottom wall of the groove 313 forms the sensing region 1, and the sensing region 1 has the metal layer 2. In a preferred embodiment, the metal layer 2 is a gold (Au), silver (Ag), aluminum (Al) or copper (Cu) thin film, and the metal layer 2 has a thickness falling within a range from 40 nm to 60 nm.

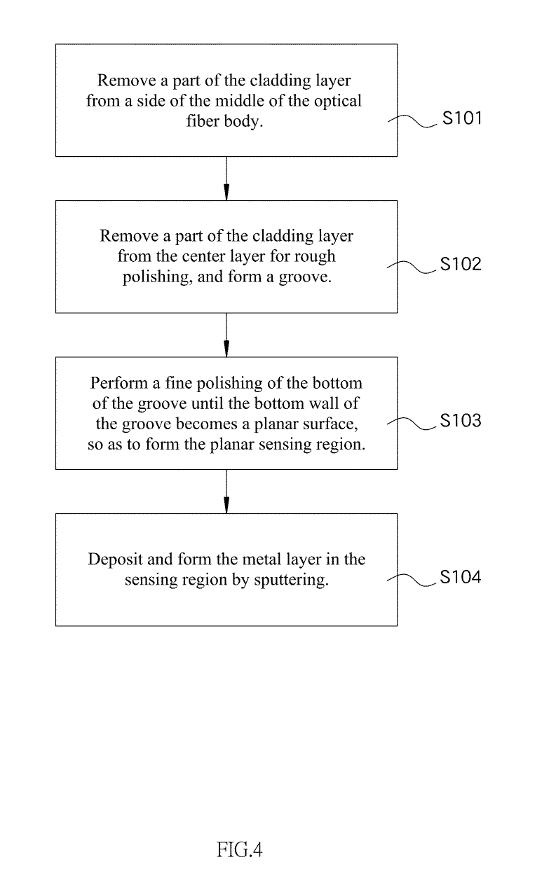

In an embodiment of the manufacturing method of the apparatus for detecting the content of oleic acid by optical fiber, a side-polished technique is adopted to carry out the steps S101 to S104 as shown in FIG. 4.

S101: Remove a part of the cladding layer 312 from a side of the middle of the optical fiber body 31.

S102: Remove a part of the cladding layer 312 from the center layer 311, and use diamond films of 30 μm grain size to conduct a rough polishing, so as to form the groove 313, wherein the groove 313 at the center layer 311 has a depth approximately equal to half of the diameter of the center layer 311.

S103: Perform a fine polishing of the bottom of the groove 313 by diamond films with a grain size of 3 μm and 1 μm sequentially, such that the bottom wall of the groove 313 is a planar surface, so as to form the planar sensing region 1.

S104: Deposit and form the metal layer 2 in the sensing region 1 by direct current (DC) sputtering.

In this preferred embodiment, the cladding layer 312 has an external diameter R of 125 μm; the center layer 311 has an external diameter r of 62.5 μm; the groove 313 has an axial length L of 5 mm; and the groove 313 has a depth D of 62.5 μm.

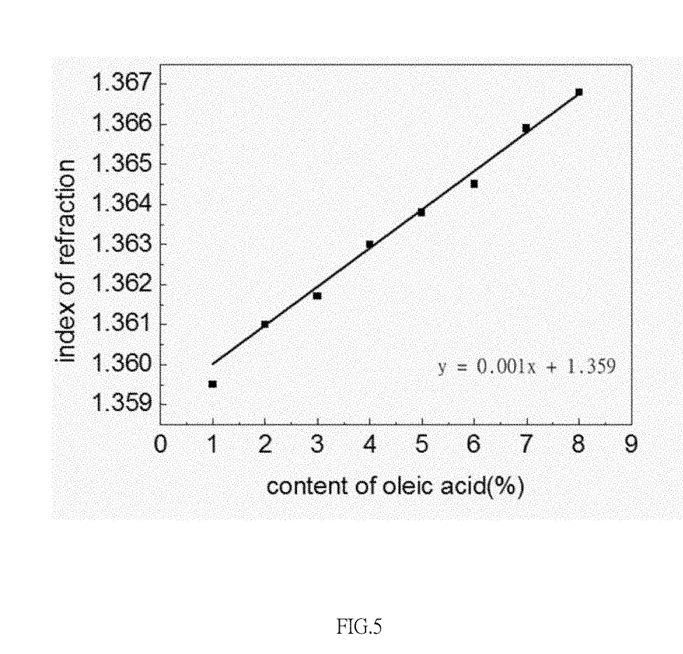

In the preparation of the analyte, the alcohol solvent and the oleic acid can be dissolved by each other, but not dissolved with oils, so that ethanol is used as a solvent and oleic acid is used as a solute to manufacture the analyte in this preferred embodiment. In FIG. 5, this preferred embodiment uses a refractometer (ATAGO R-5000) to analyze and measure the analyte with different concentration of oleic acids, and its index of refraction ranges from 1.360 to 1.367, so that we know the concentration of oleic acid in percentage by volume falls within a range from 1% to 8%. If the concentration of oleic acid is increased by 1%, then the index of refraction will be increased by 0.001 accordingly. Obviously, the change of concentration of the analyte is sensitive to the index of refraction, and thus the invention complies with the elements of the Surface Plasmon Resonance (SPR) detection.

S002: Apply a transverse magnetic wave of a light beam to the sensing region through the light guide medium 3.

As the surface plasma wave is excited by TM mode of electromagnetic wave, the interface between the metal layer 2 and the dielectric material generates the vertical component of discontinuous electric field. It makes the electrons from the metal collectively generate the oscillation of electric dipole. Therefore, the intensity of the reflected light and phase would be changed according to the analyte, and the electrons from the metal collectively produce generate the oscillation of electric dipole. When evanescent waves excite surface plasma waves, surface electrons are formed at an interface between the metal layer 2 of a specific thickness and the analyte, so as to form a coherency resonance phenomenon which is called SPR.

In a specific preferred embodiment, the light beam with a wavelength of a halogen white light falling within a range from 400 nm to 1800 nm is incident from an end of the center layer 311.

S003: Let the light beam excite the metal layer 2 and the analyte to produce a plasmon resonance.

In this preferred embodiment, the light beam is incident from an end of the center layer 311 and transmitted to the sensing region 1. Since the light is transmitted by the optical fiber body 31, therefore there is no need of calibrating the SPR resonant angle to project the light onto the sensing region 1 in order to generate a plasmon resonance phenomenon.

The optical fiber body 31 is a plastic optical fiber or a glass optical fiber, and most plastic optical fibers are multimode optical fibers with transmissions of light beams at different angles therein, so that if there is a slight bending or movement, the internal light transmission will be affected to result in a lower stability. Preferably, the optical fiber body 31 is a glass optical fiber with a stability of an internal light transmission higher than the stability of the plastic optical fiber, and thus the glass optical fiber is more advantageous for control and measurement analysis of the light beam. However, such arrangement is provided as an example for the illustrating the present invention only, but not intended for limiting the scope of the invention.

S004: Measure at least one physical property of the plasmon resonance.

In this preferred embodiment, an optical spectrum analyzer (such as the ANDO AQ-6315A spectrum analyzer, not shown in the figure) is used as a receiver to measure the physical property, wherein the physical property of the plasmon resonance is the resonant wavelength when the plasmon resonance occurs.

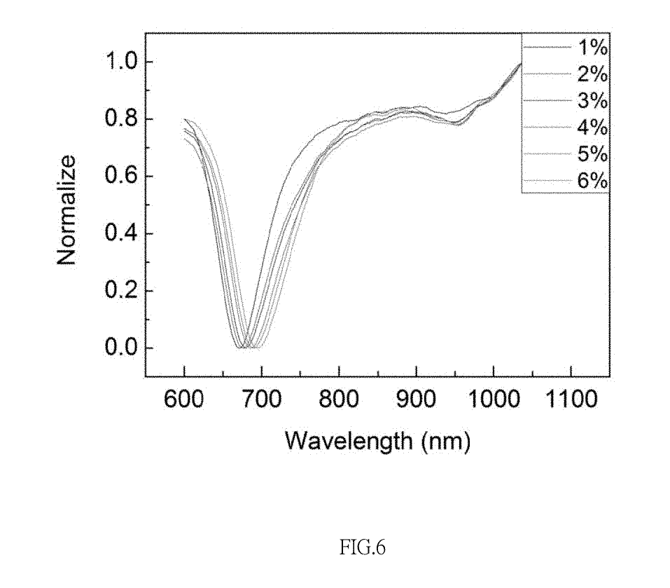

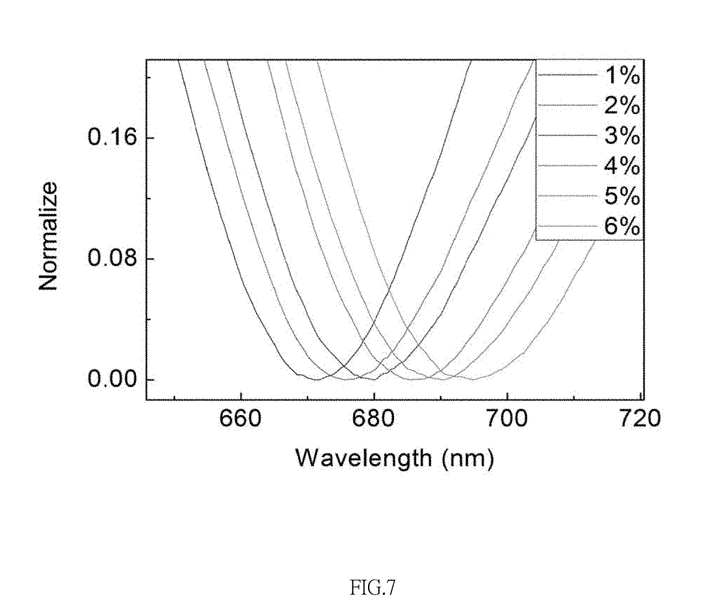

FIG. 6 shows the SPR spectrum of an analyte at different concentration of oleic acids from 0% to 6% with an increment of 1%, wherein the X-axis represents the resonant wavelength and the Y-axis represents a normalized light intensity. When the plasmon resonance occurs, the light intensity drops to 0 rapidly, so that the resonant wavelength can be used as an actual detection value when the light intensity is 0. In FIG. 7, the spectrum shown in FIG. 6 is partially enlarged when the light intensity approaches 0, and recorded as shown in Table 1 below.

TABLE 1

| | Concentration of oleic acid (%) | | | | | | |
|---|---|---|---|---|---|---|---|
| | 0 | 1 | 2 | 3 | 4 | 5 | 6 |
| Resonant | 667.5 | 671.5 | 675.5 | 680.0 | 685.5 | 690.5 | 695.0 |
| Wavelength | | | | | | | |
| λ(nm) | | | | | | | |
| Change of | | 4 | 4 | 4.5 | 5.5 | 5 | 4.5 |
| Wavelength | | | | | | | |
| Δλ (nm) | | | | | | | |

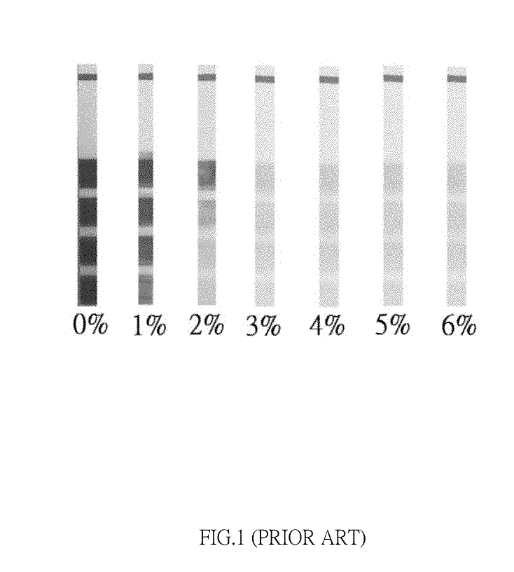

S005: Use a physical property to define the concentration of oleic acid of the analyte. Table 1 clearly shows a linear relation between the concentration of oleic acid and the resonant wavelength λ, and the linear relation can be used to quantify the concentration of oleic acid and the resonant wavelength λ, or the change of wavelength Δλ of the resonant wavelength. By measuring the resonant wavelength λ or the change of wavelength Δλ, the concentration of oleic acid of the analyte can be derived accurately. Compared with the detection result obtained by the conventional acid value test paper as shown in FIG. 1, the present invention definitely has a higher accuracy and a wider detection range.

The present invention breaks through the prior art and definitely achieves the intended effects, and the invention is novel and is not apparent to or easily perceived by persons having ordinary skill in the art. In addition, the present invention is novel, inventive, useful, and in compliance with patent application requirements, and thus is duly filed for patent application.

While the ideas and technical characteristics of the present invention have been described by means of specific embodiments, numerous modifications and variations could be made thereto by those skilled in the art without departing from the scope and spirit of the invention set forth in the claims.

1. A method for detecting a content of oleic acid by optical fiber, comprising:
setting a metal layer in a sensing region;
setting an analyte solution including oleic acid and an alcohol solvent onto the metal layer;
applying a transverse magnetic wave of a light beam into the sensing region through a light guide medium to thereby excite the metal layer and the analyte solution to produce a plasmon resonance;
measuring at least one physical property of the plasmon resonance; and
determining a concentration of oleic acid of the analyte solution according to the physical property.

2. The method for detecting the content of oleic acid by optical fiber according to claim 1, wherein the metal layer is a gold (Au), silver (Ag), aluminum (Al) or copper (Cu) thin film.

3. The method for detecting the content of oleic acid by optical fiber according to claim 2, wherein the metal layer has a thickness falling within a range from 40 nm to 60 nm.

4. (canceled)

5. The method for detecting the content of oleic acid by optical fiber according to claim 1, wherein the alcohol solvent of the analyte solution is ethanol.

6. The method for detecting the content of oleic acid by optical fiber according to claim 1, wherein the light beam has a wavelength falling within a range from 400 nm to 1800 nm before the light beam is incident onto the sensing region.

7. The method for detecting the content of oleic acid by optical fiber according to claim 1, wherein the physical property of the plasmon resonance is measured by an optical spectrum analyzer.

8. The method for detecting the content of oleic acid by optical fiber according to claim 1, wherein:
the light guide medium includes an optical fiber body,
the optical fiber body includes a center layer and a cladding layer coaxial with the center layer,
a groove is formed on a side of the middle of the optical fiber body and sunken into the center layer,
a bottom wall of the groove forms the sensing region, and
the light beam is incident from an end of the center layer and transmitted to the sensing region.

9. The method for detecting the content of oleic acid by optical fiber according to claim 8, wherein the bottom wall of the groove is a planar surface.

10. The method for detecting the content of oleic acid by optical fiber according to claim 8, wherein the metal layer is formed at the sensing region by sputtering.

11. The method for detecting the content of oleic acid by optical fiber according to claim 1, wherein the physical property of the plasmon resonance is the resonant wavelength when the plasmon resonance occurs.

12. An apparatus for detecting a content of oleic acid by optical fiber comprising a light guide medium, the light guide medium having an optical fiber body, the optical fiber body having a center layer and a cladding layer coaxial with the center layer, a groove being formed on a side of a middle of the optical fiber body and sunken into the center layer, a bottom wall of the groove forming a sensing region, the sensing region having a metal layer set therein, wherein the apparatus is configured to:
receive an analyte solution including oleic acid and an alcohol solvent onto the metal layer; and
apply a transverse magnetic wave of a light beam into the sensing region through the light guide medium to thereby excite the metal layer and the analyte solution to produce a plasmon resonance,
at least one physical property of the plasmon resonance measured to thereby determine a concentration of oleic acid of the analyte solution according to the physical property.

13. The apparatus for detecting the content of oleic acid by optical fiber according to claim 12, wherein the bottom wall of the groove is a planar surface.

14. The apparatus for detecting the content of oleic acid by optical fiber according to claim 12, wherein the metal layer is formed at the sensing region by sputtering.

15. The apparatus for detecting the content of oleic acid by optical fiber according to claim 12, wherein the groove at the center layer has a depth approximately equal to half of the diameter of the center layer.

16. The apparatus for detecting the content of oleic acid by optical fiber according to claim 12, wherein the cladding layer has an external diameter of 125 μm, and the center layer has an external diameter of 62.5 μm, and the groove has an axial length of 5 mm, and the groove has a depth of 62.5 μm.

17. The apparatus for detecting the content of oleic acid by optical fiber according to claim 12, wherein the optical fiber body is a glass optical fiber.

18. A method of manufacturing the apparatus for detecting the content of oleic acid by optical fiber according to claim 12, comprising:
removing a portion of the cladding layer from the side of the middle of the optical fiber body;
performing a rough polishing of a portion of the center layer exposed by the removal of the portion of the cladding layer to thereby form the groove;
performing a fine polishing of the bottom wall of the groove to form the sensing region, the sensing region being planar; and
depositing and forming the metal layer at the sensing region by sputtering.

19. A method for detecting a content of oleic acid by optical fiber, comprising:
providing a light guide medium including an optical fiber body, the optical fiber body including a center layer and a cladding layer coaxial with the center layer, a groove being formed on a side of the middle of the optical fiber body and sunken into the center layer, a bottom wall of the groove forming a sensing region;
setting a metal layer in the sensing region of the light guide medium;
setting an analyte solution including oleic acid and an ethanol solvent onto the metal layer;
transmitting a light beam to the sensing region to thereby excite the metal layer and the analyte solution to produce a plasmon resonance, the light beam being incident from an end of the center layer of the optical fiber body, the light beam having a wavelength falling within a range from 400 nm to 1800 nm before the light beam is incident onto the sensing region;
measuring a resonant wavelength when the plasmon resonance occurs; and
determining a concentration of oleic acid of the analyte solution according to the resonant wavelength.
